(19) United States
(12) Patent Application Publication
(10) Pub. No.: US 2008/0283066 A1
(43) Pub. Date: Nov. 20, 2008

(54) DELIVERY DEVICE FOR IMPLANTABLE SENSORS

(75) Inventors: Jessie Delgado, Murrieta, CA (US); John S. Greenland, San Diego, CA (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 12/103,585

(22) Filed: Apr. 15, 2008

Related U.S. Application Data
(60) Provisional application No. 60/938,562, filed on May 17, 2007.

(51) Int. Cl. A61B 17/94

(52) U.S. Cl. 128/899; 606/1

(57) ABSTRACT

A delivery system for an implantable medical device including a tether retaining feature having a bore. The system includes a connector having a top surface, a bottom surface, a rail extending in a proximal direction from the connector, and an aperture sized to receive the tether retaining feature and reduce movement of the connector with respect to the implantable medical device in a plane parallel to the aperture. A tether is sized to fit within the bore of the tether retaining feature. The tether acts against the top surface of the connector to retain the bottom surface of the connector proximal to the implantable medical device when the tether is located within the bore of the tether retaining feature. A method for delivering an implantable medical device including a tether retaining feature.

### CROSS-REFERENCE TO RELATED APPLICATIONS

This application claims priority to U.S. Provisional Application No. 60/938,562, filed May 17, 2007, entitled “DELIVERY DEVICE FOR IMPLANTABLE SENSORS.” This application is also related to U.S. Provisional Patent Application No. 60/844,953, filed Sep. 15, 2006, entitled “DELIVERY SYSTEM FOR AN IMPLANTABLE PHYSIOLOGICAL SENSOR;” and U.S. Provisional Patent Application No. 60/844,821, filed Sep. 15, 2006 entitled “ANCHOR FOR AN IMPLANTABLE SENSOR.” All three of the above applications are herein incorporated by reference in their entirety.

### TECHNICAL FIELD

The present invention relates to medical devices and methods for anchoring implantable medical devices in the body. In particular, the present invention is a delivery system for releasably coupling to an implantable medical device during delivery and deployment.

### BACKGROUND

Medical devices are known that can be implanted within a patient's body for monitoring one or more physiological parameters and/or for providing therapeutic functions. For example, sensors or transducers can be placed in the body for monitoring a variety of properties, such as temperature, blood pressure, strain, fluid flow, chemical properties, electrical properties, magnetic properties, and the like. In addition, medical devices can be implanted that perform one or more therapeutic functions, such as drug delivery, cardiac pacing, defibrillation, electrical stimulation, and the like.

One parameter of particular interest is blood pressure. One or more implantable pressure sensing modules can be used in conjunction with cardiac rhythm management (CRM) devices to facilitate optimization of CRM device settings. In such systems, the pressure sensing module is delivered transvenously to a target vessel (e.g., the pulmonary artery) and anchored in the vessel using various fixation techniques. Accurate placement of the sensing module is an important factor in accurately and reliably measuring the desired parameter. Additionally, under some circumstances, it becomes necessary to re-position an implantable sensor module after initial deployment or, alternatively, to remove the sensor from the patient entirely.

Thus, a need exists for apparatus and methods for accurately delivering and deploying implantable medical devices within a patient's body. In particular, there is a need for a mechanism for releasably engaging an implantable sensor to facilitate accurate deployment of the sensor at a desired implantation site.

### SUMMARY

In one embodiment, the invention is a delivery system for an implantable medical device including a tether retaining feature having a bore. The system comprises a connector having a top surface, a bottom surface, a rail extending in a proximal direction from the connector, and an aperture sized to receive the tether retaining feature and reduce movement of the connector with respect to the implantable medical device in a plane parallel to the aperture. A tether is sized to fit within the bore of the tether retaining feature. The tether acts against the top surface of the connector to retain the bottom surface of the connector proximal to the implantable medical device when the tether is located within the bore of the tether retaining feature.

In another embodiment, the invention is a delivery system for an implantable medical device including a tether retaining feature. The system comprises a connector having an aperture sized to receive the tether retaining feature and a tether sized to fit within the tether retaining feature to releasably couple the connector to the implantable medical device.

In another embodiment, the invention is a method for delivering an implantable medical device including a tether retaining feature. The method comprises inserting the tether retaining feature into an aperture of a connector. The connector is releasably coupled to the implantable medical device by inserting a tether into the tether retaining feature. The implantable medical device is positioned within a patient. An anchor coupled to the implantable medical device is deployed. The connector is released from the implantable medical device by sliding the tether through a bore of the tether retaining feature. The connector is then removed.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a delivery system for delivering an implantable medical device, which in the illustrated embodiment is an implantable sensor assembly, to an implantation site within a pulmonary artery of a heart according to one embodiment of the present invention.

FIG. 2 is a partial cutaway perspective view of the distal portion of the delivery system of FIG. 1.

FIGS. 3-5 are partial cross-sectional views of the distal portions of an inner member and a retaining element of the delivery system of FIG. 1.

FIG. 6 is a partial cutaway view of a distal portion of an implantable sensor delivery system according to another embodiment of the present invention.

FIGS. 7-10 are perspective views illustrating a sensor assembly being deployed using the implantable sensor assembly delivery system of FIG. 6.

FIGS. 11-12 illustrate a distal portion of a delivery system for an implantable medical device according to another embodiment of the present invention.

FIG. 13 illustrates a distal portion of a delivery system for an implantable medical device according to yet another embodiment of the present invention.

FIGS. 14A-14B illustrate an inner member adapted for use in conjunction with the delivery systems of FIGS. 11-13 according to one embodiment of the present invention.

FIG. 15 illustrates an exemplary method of using the delivery systems of FIGS. 11-13.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

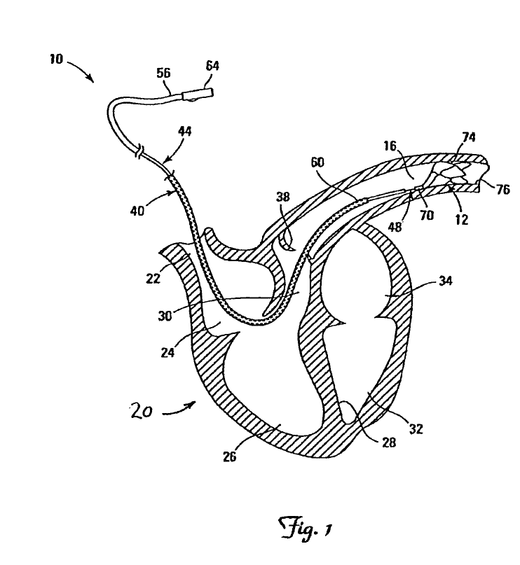

FIG. 1 shows a delivery system 10 for delivering an implantable medical device, which in the illustrated embodiment is an implantable sensor assembly 12, to a target implantation site within a pulmonary artery 16 of a heart 20 according to one embodiment of the present invention. As shown, the heart 20 generally includes a superior vena cava 22, a right atrium 24, a right ventricle 26, a ventricular septum 28, a right ventricular outflow tract 30, a left ventricle 32 and a left atrium 34. As shown, the right ventricular outflow tract 30 leads to the pulmonary artery 16, which is separated from the right ventricle 26 by a pulmonary artery valve 38.

The delivery system 10 is sized (i.e., has a length and diameter) to navigate the patient's vasculature to the target implantation site from a location external to the patient's body. In the illustrated embodiment, the delivery system 10 enters the heart 20 through the superior vena cava 22, and extends through the right atrium 24 and the right ventricular outflow tract 30 to deliver the implantable sensor assembly 12 in the main pulmonary artery 16. In such an embodiment, the delivery system 10 may be transvenously advanced to the heart 20 by any methods known in the art. For example, as is well known, the delivery system 10 may enter the patient's vasculature system through a percutaneous incision into the left subclavian vein, the left auxiliary vein, the left internal or external jugular vein, the left brachiocephalic vein, or through a femoral approach. In various embodiments, the delivery system 10 may be used to deliver an implantable sensor assembly 12 to a branch of the pulmonary artery 16 (e.g., the right or left pulmonary artery, not shown). In other embodiments, the delivery system 10 may be used to deliver an implantable sensor assembly to other areas of the patient's vasculature.

As shown in FIG. 1, the delivery system 10 includes a flexible, elongate outer catheter 40, a flexible, elongate inner member 44 disposed within the outer catheter 40, and a flexible, elongate retaining element 48 disposed within the inner member 44 and releasably engaged with the sensor assembly 12. The outer catheter 40 includes a proximal end 56 and a distal end 60. As will be appreciated, the outer catheter 40 includes at least one lumen (not shown in FIG. 1) through which the inner member 44 is disposed. As will be explained in detail below, the delivery system 10, and other embodiments of the present invention, advantageously provide accurate control over the implantation location of the sensor assembly 12. Additionally, the delivery systems of the present invention allow the physician to re-position and re-deploy the sensor assembly 12 if necessary or desired.

The outer catheter 40 and the inner member 44 are movable relative to each other, and the retaining element 48 is movable relative to the inner member 44, to deploy the sensor assembly 12 at the target implantation site. In the illustrated embodiment, the delivery system 10 includes a control mechanism 64 on the proximal end 56 of the outer catheter 40 and which is operatively coupled to at least the inner member 44. The control mechanism 64 is operable to allow a physician to control relative movement of at least the outer catheter 40 and inner member 44, and in some embodiments, the retaining element 48, for delivery and deployment of the sensor assembly 12. The control mechanism 64 may include any mechanism or structure known or later developed for controlling the relative longitudinal and/or rotational movement of inner and outer catheters of a dual catheter system. In one exemplary embodiment, the control mechanism 64 includes a thumbwheel operatively coupled to the inner member 44 to permit the physician to slide the inner member 44 within the outer catheter 40.

The outer catheter 40 can be any catheter known in the art or later developed for accessing a target implantation location in a patient's vasculature. As will be appreciated, the particular design and construction, including materials, of the outer catheter 40 is determined based on the needs of the patient, and in particular, the selected implantation location for the implantable sensor assembly 12. In one embodiment, the outer catheter 40 is a catheter configured for accessing the pulmonary artery 16 or a branch thereof. In one embodiment, the outer catheter 40 can be advanced to the pulmonary artery 16 over a guidewire positioned therein through a Swan Ganz procedure, in which a balloon catheter is inserted into the venous system and floated with the blood flow into and through the heart 20 out to the pulmonary artery 16.

As shown in FIG. 1, the sensor assembly 12 includes an implantable sensor 70 and an anchor 74 coupled to the sensor 70. As will be discussed in more detail below, the anchor 74 is an expandable structure configured to assume a collapsed configuration for transvenous delivery of the sensor assembly 12 to the desired implantation location through the delivery system 10, and an expanded configuration, illustrated in FIG. 1, in which the anchor 74 engages an inner surface 76 of the pulmonary artery 16.

The sensor 70 may be configured to perform one or more designated functions, which may include taking one or more physiological measurements. The sensor 70 may be configured to measure any known physiologic parameters such as, for example, blood pressure, temperature, blood or fluid flow, strain, electrical, chemical, or magnetic properties within the body. The specific parameters to be measured, and thus the implantation site for the sensor assembly 12, are determined based on the particular therapeutic needs of the patient. In one exemplary embodiment, the sensor 70 may be configured to measure blood pressure in the pulmonary artery 16 (as illustrated in FIG. 1). In one embodiment, the sensor 70 may further be adapted to store and/or transmit blood pressure data to another implanted device (e.g., a cardiac rhythm management device such as a pacemaker, not shown) and/or a device (e.g., a monitor or programmer) located external to the patient's body.

In various embodiments, the sensor 70 is configured to communicate with other devices, such as an external device or another implantable medical device (e.g., a pacemaker and/or defibrillator) via a wireless communication link. Various types of wireless communication circuitry are well known in the art, and the specific type and/or style of wireless communication that can be used is not limited. For example, ultrasonic waves, acoustic communications, radio frequency communications, and the like may be used. In one embodiment, the sensor 70 includes an acoustic transmitter/receiver configured for acoustic telemetry.

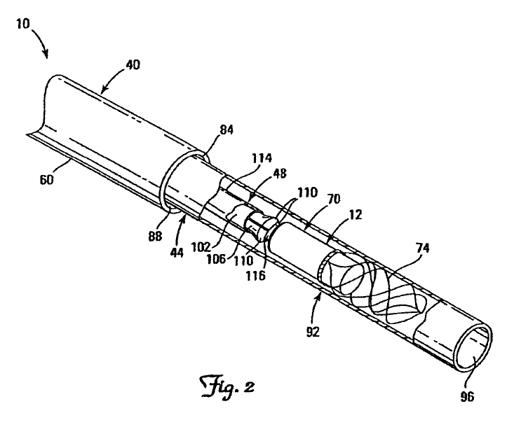

FIG. 2 is a perspective view of the distal portion of the delivery system 10 showing a partial cutaway of the inner member 44, and further showing the implantable sensor assembly 12 releasably coupled to the retaining element 48 for delivery of the sensor assembly 12. As shown in FIG. 2, the outer catheter 40 includes a lumen 84 sized to slidably receive the inner member 44, and terminates in a distal opening 88. As further shown in FIG. 2, the inner member 44 includes a distal end portion 92 in the form of a sheath having a distal opening 96 and an inner diameter and length sized to receive the sensor assembly 12 so as to maintain the anchor 74 of the sensor assembly 12 in a collapsed configuration during delivery.

As can further be seen in FIG. 2, the retaining element 48 includes a body 102 having a distal end 106, a plurality of deflectable jaw members 110 extending distally from the distal end 106, and a tubular actuating member 114 (shown in cutaway view to illustrate the body 102) slidably disposed over the body 102. The jaw members 110 operate as a sensor engagement structure for releasably engaging a portion of the sensor 70. As will be explained in more detail below, the jaw members 110 are naturally biased radially outwardly in an undeflected state, and the actuating member 114 is configured to force the jaw members 110 radially inward so as to engage the sensor assembly 12 by clamping onto the sensor assembly 12.

In the illustrated embodiment, the sensor 70 includes a hub 116 at its proximal end. As shown, the hub 116 is configured to mate with the jaw members 110 to promote positive coupling of the retaining element 48 and the sensor 70. In other embodiments, a different engagement feature may be included on the sensor 12. In other embodiments, the hub 116 or other engagement feature may be omitted.

In various embodiments, the retaining element 48 may include different sensor engagement structures. For example, in one embodiment, the retaining element 48 may include an elongated tether having a hook at its distal end, which hook is adapted to engage an aperture or loop on the sensor 70. Other embodiments may incorporate still other sensor engagement structures. In still other embodiments, the retaining element 48 is simply a solid or tubular structure (i.e., lacks the jaw members 110 and actuating member 114), and can be used to push the sensor assembly 12 distally and/or resist proximal displacement of the sensor assembly 12.

The inner member 44 and the retaining element 48 are dimensioned so as to extend proximally from the implantation location (e.g., a location within the pulmonary artery 16 as shown in FIG. 1) to or near the proximal end 56 of the outer catheter 40. Additionally, as shown in FIG. 2, the outer catheter 40 can be retracted proximally relative to the inner member 44, or alternatively, the inner member 44 (with the sensor assembly 12 retained therein) can be advanced distally relative to the outer catheter 40, such that the sensor assembly 12 may be deployed from the distal opening 96 of the inner member 44 without interference from the outer catheter 40.

The outer catheter 40 is sized to accommodate the selected implantable sensor assembly 12 (or other implantable device), and as will be appreciated, has a length sufficient to transvenously deliver the sensor assembly 12 to the desired implantation site through a percutaneous access site such as described above. In various exemplary embodiments, the outer catheter 40 may range in size from a 6 French to a 20 French guide catheter. In some embodiments, for example, where the sensor assembly 12 is configured for implantation in the pulmonary artery 16, the outer catheter 40 may range in size from 10 French to 16 French.

The inner member 44 may be made from substantially the same or identical materials as the outer catheter 40. In some embodiments, the inner member 44 may be made substantially from a braided composite tubing as is known in the art for catheters and the like. In some embodiments, the distal end portion 92 of the inner member 44 may be made from a relatively low durometer material such as, for example, low-durometer Pebax. In other embodiments, the inner surface of the distal end portion 92 may include a biocompatible, lubricious coating to facilitate relative displacement of the inner member 44 and the sensor assembly 12 without undue friction.

The materials selected for the retaining element 48 are not of particular significance. In some embodiments, the body 102 and/or the actuating member 114 may be made from a metal (e.g., stainless steel) or a polymeric material. In some embodiments, the jaw members 110 may be made from materials exhibiting shape memory and/or superelastic properties, such as, for example, Nitinol or any of a number of other shape memory alloys or polymers. In some embodiments, the retaining element 48 may include a radio-opaque marker at or near its distal end.

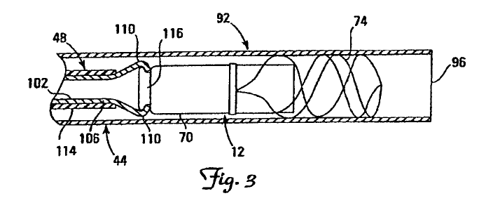

FIGS. 3-5 are partial cross-sectional views of the distal portions of the inner member 44 and the retaining element 48 illustrating the deployment of the sensor assembly 12 from the inner member 44 according to one embodiment of the present invention. It will be appreciated that the outer catheter 40 has already been retracted proximally relative to the inner member 44, such as is shown in FIG. 2. As shown in FIG. 3, the sensor assembly 12 is initially fully retained within the distal end portion 92 of the inner member 44, with the anchor 74 in the collapsed configuration. As further shown in FIG. 3, the actuating member 114 of the retaining element 48 is positioned at least partially over the jaw members 110, thereby clamping the jaw members 110 onto the proximal hub 116 of the sensor 70. As explained above, however, in other embodiments, the jaw members 110 may engage other engagement features of the sensor assembly 12. Alternatively, the engagement feature may be omitted, and the jaw members 110 may engage other portions of the sensor assembly 12 (e.g., the housing of the sensor 70 or a portion of the anchor 74).

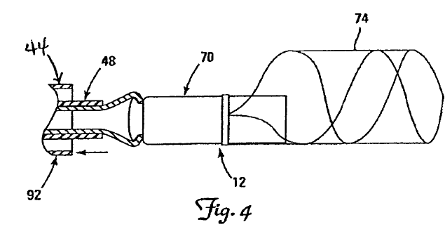

In FIG. 4, the inner member 44 has been moved proximally relative to the sensor assembly 12 so as to release the sensor assembly 12 (or at a minimum, the anchor 74) from the distal end portion 92 of the inner member 44. With the inner member 44 so positioned, the anchor 74 is permitted to expand to an expanded configuration for frictionally engaging an inner surface of the target vessel (e.g., the pulmonary artery, see FIG. 1) to secure the sensor assembly 12 therein. The anchor 74 may be a self-expanding anchor having a stent-like structure similar to known cardiovascular stents. Alternatively, the anchor 74 may be expandable by other means (e.g., by a balloon). In various embodiments, the anchor 74 may be any of the anchoring structures disclosed in co-pending and commonly assigned U.S. patent application Ser. No. 11/216,738, entitled “DEVICES AND METHODS FOR POSITIONING AND ANCHORING IMPLANTABLE SENSOR DEVICES,” filed Aug. 31, 2005, and U.S. Provisional Patent Application No. 60/844,821, entitled “ANCHOR FOR AN IMPLANTABLE SENSOR,” filed on Sep. 15, 2006. The contents of the foregoing pending applications are both herein incorporated by reference in their entirety.

As shown in FIG. 4, the retaining element 48 can remain coupled to the sensor assembly 12 after deployment of the anchor 74 from the distal end portion 92 of the inner member 44. This permits the sensor assembly 12 to be repositioned to another location within the target vessel, or another area of the patient's vasculature, if desired. For example, it may be desirable to perform various diagnostic tests on the sensor 70 to confirm that it is functioning properly and/or that the chosen implantation location is suitable. Alternatively, or additionally, the physician may wish to confirm that the sensor assembly 12 is sufficiently secured at the implantation site before releasing the retaining element 48. In particular, where the anchor 74 is one of the re-positionable anchor structures disclosed in co-pending and commonly assigned U.S. Provisional Patent Application No. 60/844,821 titled “ANCHOR FOR AN IMPLANTABLE SENSOR”, the sensor assembly 12, including the anchor 74, can be retracted within the distal end portion 92 of the inner member 44 by pulling proximally on the retaining element 48 while holding the inner member 44 in place. The inner member 44, with the sensor assembly 12 retained therein, can then be re-positioned within the target vessel, and the sensor assembly 12 re-deployed as described above. Alternatively, the inner member 44 may be retracted back within the outer catheter 40 (see FIG. 2), and the entire delivery system can be re-located to a different target implantation site, or can be removed from the patient entirely.

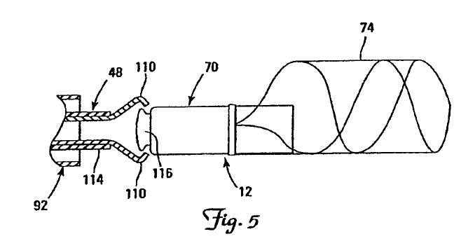

FIG. 5 illustrates the sensor assembly 12 after being de-coupled from the retaining element 48. As shown in FIG. 5, with the actuating member 114 retracted proximally, the jaw members 110 are allowed to resume their undeflected configuration and disengage from the hub 116.

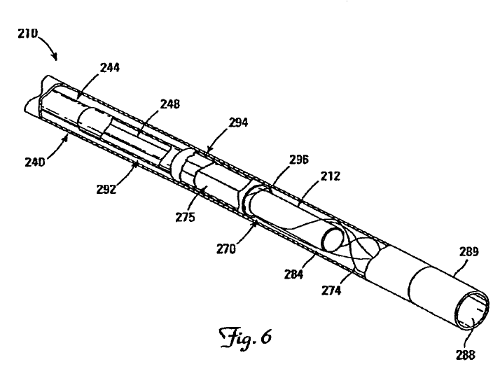

FIG. 6 is a partial cutaway view of a distal portion of an implantable sensor delivery system 210 and an implantable sensor assembly 212 coupled thereto according to another embodiment of the present invention. As shown in FIG. 6, the delivery system 210 includes an elongate outer catheter 240, an elongate inner member 244, and an elongate retaining element 248. As further shown in FIG. 6, like the sensor assembly 12 described above, the sensor assembly 212 includes a sensor element 270 and an anchor portion 274. In the illustrated embodiment, the sensor 270 includes a proximal portion 275 releasably engaged by and received by the inner member 244.

As shown, the outer catheter includes a lumen 284 sized to slidably receive the inner member 244, and terminates in a distal opening 288. The outer catheter 240 may be of substantially the same construction as the outer catheter 40 described above. In the illustrated embodiment, the outer catheter 240 includes a radio-opaque end portion 289, which may optionally include an atraumatic tip. In other embodiments, the radio-opaque portion 289 is omitted.

As further shown in FIG. 6, the inner member 244 is generally tubular and includes a distal end portion 292 including a socket 294 having a distal opening 296 and an inner diameter and length sized to receive and frictionally engage at least a portion, (i.e., in the illustrated embodiment, the proximal portion 275) of the sensor 270. Thus, unlike the distal end portion 92 of the inner member 44 described above, the distal end portion 292 is not sized to receive the entire sensor assembly 212, and in particular, the anchor portion 274 of the sensor assembly 212. Rather, in the embodiment illustrated in FIG. 6, the anchor portion 274 is retained in its collapsed configuration for delivery by the outer catheter 240. The outer catheter 240 and/or the inner member 244 may include at or near their proximal ends (not shown) a control mechanism similar or identical to those described above in connection with the delivery system 10.

In one embodiment, the sensor proximal end portion 275 may be held within the socket 294 by an interference fit. In such embodiments, the inner diameter of the socket 294 may be sized to be from about 0.002 inches to about 0.004 inches smaller than the outer diameter of the sensor proximal end portion 275, to ensure sufficient frictional engagement of the sensor 270 during delivery. In another embodiment, a relatively weak adhesive bond may be utilized to releasably retain the sensor proximal end portion 275 within the socket 294.

As shown, the retaining element 248 is disposed within the generally tubular inner member 244, and like the retaining element 48 described above, is adapted to releasably engage the sensor assembly 212. Thus, it will be appreciated that the retaining element 248 may be substantially the same or identical in design and/or function as the retaining element 48 described above. For example, in one embodiment, the retaining element 248 may have the same sensor engagement structure (e.g., deflectable jaw members) as the retaining element 48. Similarly, as will further be appreciated, the sensor 270, or in some embodiments, another portion of the sensor assembly 212, may include an engagement feature similar to the hub 116 of the sensor 70. In still other embodiments, the retaining element 248 may include no distal mechanism (such as the jaw members 110 of the retaining element 48), and may simply allow the physician to push the sensor assembly 212 distally, or alternatively, to resist proximal displacement of the sensor assembly 212. In short, any structure or mechanism capable of releasably engaging and retaining the sensor assembly 212 during delivery and deployment can be incorporated into the retaining element 248.

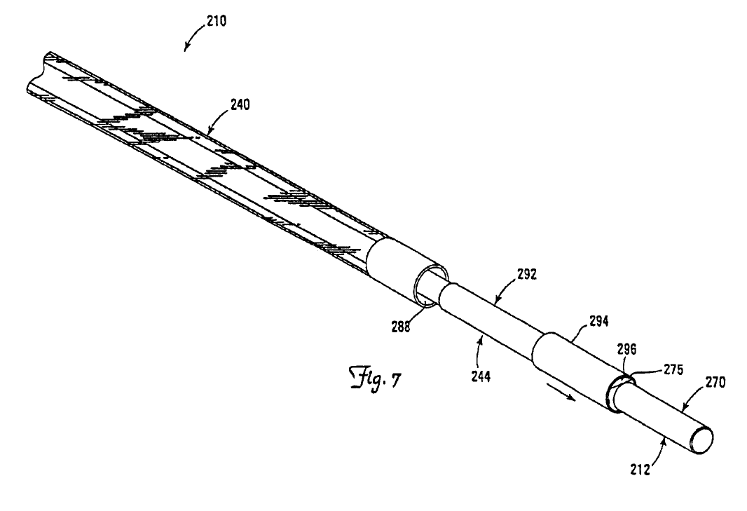

FIGS. 7-10 illustrate the sensor assembly 212 being deployed using the implantable sensor assembly delivery system 210 according to one embodiment of the present invention. For the purpose of this description only, the anchor 274 is not shown in FIGS. 7-10. It is emphasized that the sensor assembly 212 shown in FIGS. 7-10, however, may also include the anchor 274, which may be a self-expanding anchor similar or identical to those described above with respect to the anchor 74.

As shown in FIG. 7, the distal end portion 292 can be displaced distally with respect to the outer catheter 240. This can be accomplished by maintaining the outer catheter 240 in place and distally advancing the inner member 244 (e.g., by use of a control mechanism operatively coupled to one or both of the outer catheter 240 and the inner member 244). Alternatively, or additionally, the inner member 244 may be held in place while the outer catheter 240 is retracted proximally. In either case, the sensor assembly 212 can be deployed out of the distal opening 288 with the proximal portion 275 of the sensor 270 retained within the socket 294 of the inner member 244. It will be appreciated that the anchor 274 (not shown) may then be expanded, or will self-expand, upon being deployed from the distal opening 288 of the outer catheter 240.

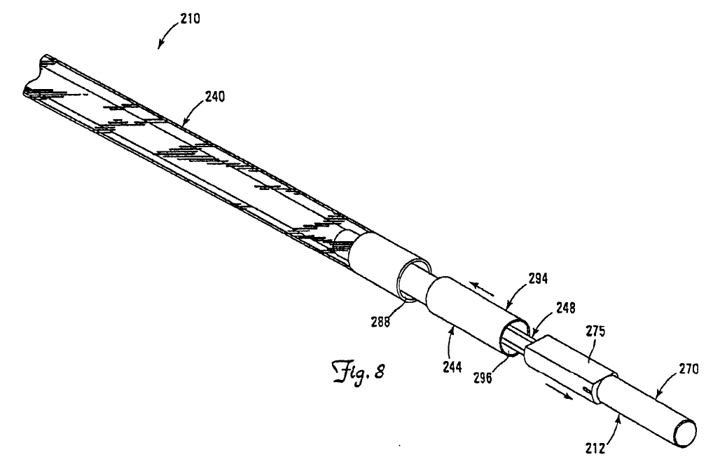

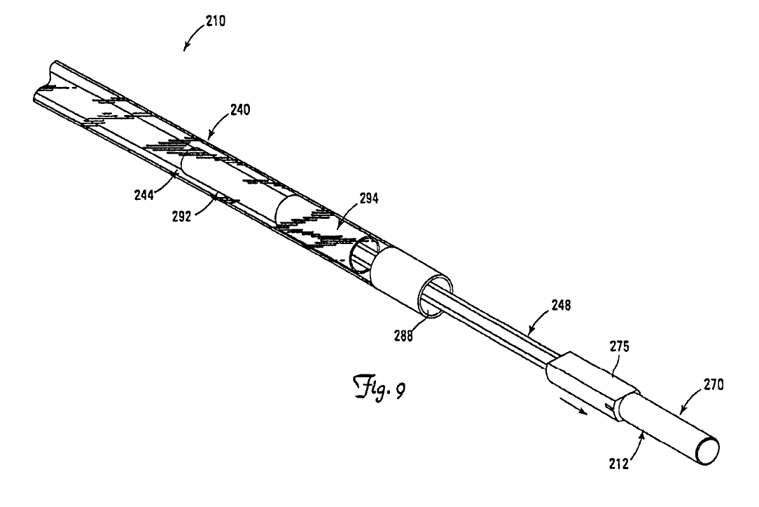

FIGS. 8-9 illustrate the delivery system 210 with the sensor assembly 212 displaced distally from the distal opening 296 of the socket 294, with the retaining element 248 still releasably coupled to the sensor 270. Such displacement can be accomplished, for example, by maintaining the sensor assembly 212 in position using the retaining element 248 and simultaneously retracting the inner member 244 (e.g., by operating a control mechanism such as a thumbwheel, not shown, coupled to the inner member 244). Alternatively, or additionally, and particularly if the anchor (not shown) has not yet significantly engaged with the target vessel tissue, the inner member 244 may be maintained in position while the retaining element 248, and accordingly, the sensor assembly 212, are pushed in the distal direction. As shown in FIG. 9, the inner member 244 can, in some embodiments, be fully retracted within the outer catheter 240 with the retaining element still coupled to the sensor 270.

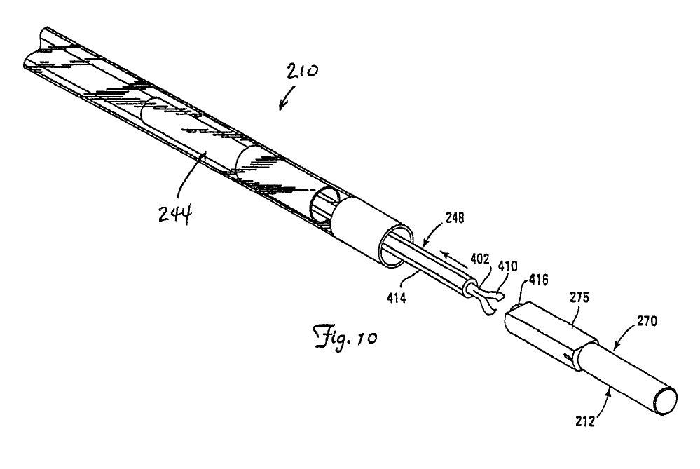

FIG. 10 illustrates the delivery system 210 with the retaining element 248 fully disengaged and de-coupled from the sensor assembly 212 and partially retracted back within the inner member 244 and outer catheter 240. In the illustrated embodiment, the retaining element 248 is shown to be substantially similar to the retaining element 48 above, and includes an inner body member 402 including a plurality of distal jaw members 410, and an outer actuating member 414 disposed over the body member 402 for causing the jaw members 410 to engage the sensor 270. Again, however, any structure or mechanism capable of releasably engaging and retaining the sensor assembly 212 as necessary for the particular deployment technique used can be incorporated into the retaining element 248.

As previously discussed, the outer catheter 240, the inner member 244, and/or the retaining element 248 may, in various embodiments, be of substantially the same or identical construction as the outer catheter 40, the inner member 44, and the retaining element 48 described above. In some embodiments, all or part of the distal end portion 292, including the socket 294, may be of a relatively low durometer material, e.g., low durometer Pebax, as compared to other portions of the inner member 244. Such configurations advantageously promote positive engagement of the sensor proximal end portion 275 within the socket 294, yet still permit the sensor 270 to be released from the socket 294 without requiring undue force.

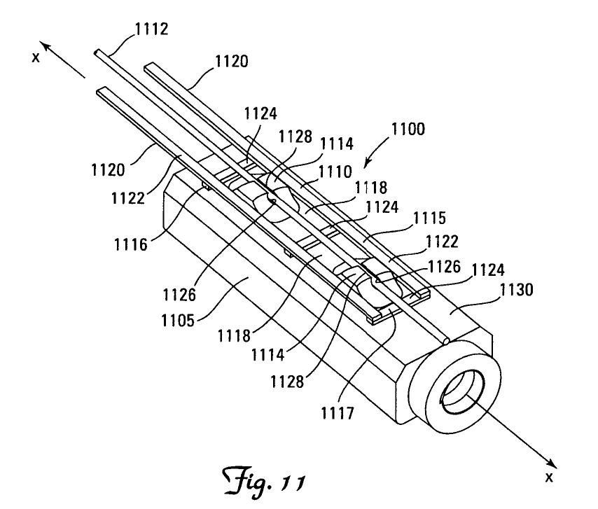

FIG. 11 is a perspective view of the distal end of a delivery system 1100 according to yet another embodiment of the present invention. In the illustrated embodiment, the delivery system 1100 includes an implantable medical device 1105, a connector 1110, a tether 1112, and tether retaining features 1114. In one embodiment, the implantable medical device 1105 is a sensor assembly. The connector 1110 includes a main portion 1115, bottom surface 1116, a top surface 1117, apertures 1118, and rails 1120. The rails 1120 extend in a proximal direction from the connector main portion 1115. Similarly, the tether 1112 extends in a proximal direction from the implantable medical device 1105.

In the embodiment shown in FIG. 11, the main portion 1115 includes side pieces 1122 and cross pieces 1124. The side pieces 1122 are located on both sides of the tether retaining features 1114 and extend proximally to form the rails 1120. In another embodiment, the rails 1120 are welded or otherwise coupled to the side pieces 1122. The cross pieces 1124 extend between the side pieces 1122, thus forming a ladder shape, as shown in FIG. 11. The configuration of the side pieces 1122 and cross pieces 1124 creates the apertures 1118 in the connector 1110. In one embodiment, the cross pieces 1124 and side pieces 1122 are formed from flat ribbon wire. In one embodiment, the flat ribbon wire has a width of approximately 0.010 inch and a thickness of approximately 0.005 inch. In another embodiment, the cross pieces 1124 and side pieces 1122 are formed from round wire. In one embodiment, the round wire has a diameter of approximately 0.007 inch. In another embodiment, the connector 1110 is manufactured from any combination of flat ribbon and round wire. In one embodiment, the wire is comprised of stainless steel or nitinol.

The apertures 1118 are sized to receive the tether retaining features 1114 and reduce movement of the connector 1110 with respect to the implantable medical device 1105 in a plane parallel to the apertures 1118. In one embodiment, the aperture 1118 has a length of about 0.40 inch and a width of about 0.20 inch. As shown in FIG. 11, the tether retaining features 1114 do not fit snugly in the apertures 1118, but instead allow some movement in a direction parallel to the longitudinal axis X-X of the connector 1110. In another embodiment, the tether retaining features 1114 fit snugly within the apertures 1118. In yet another embodiment, the apertures 1118 allow for movement of the connector 1110 in a direction other than parallel to the longitudinal axis X-X, or in addition to a direction parallel to the longitudinal axis X-X.

The tether 1112 is shown inserted into the tether retaining features 1114. The tether 1112 can comprise a substantially rigid wire, a substantially flexible wire, a suture, or any other elongated member having a size allowing it to fit within the tether retaining features 1114. In one embodiment, the tether 1112 comprises 304 grade stainless steel. As shown in FIG. 11, the tether retaining features 1114 each have a bore 1126 and an opening 1128. The bore 1126 and the tether 1112 are sized to allow the tether 1112 to slide within the bores 1126. In one embodiment, the fit between the bore 1126 and the tether 1112 is an interference fit. In one embodiment, the tether 1112 has a diameter of about 0.010 inch and the bore has a diameter of about 0.010 inch. In another embodiment, the tether 1112 slides loosely within the bore 1126. In one embodiment, the opening 1128 has a size of about 0.004 inch. In one embodiment, the tether retaining features 1114 are integral with the implantable medical device 1105. In another embodiment, the tether retaining features 1114 are coupled to the implantable medical device 1105. In one embodiment, the tether retaining features 1114 are welded to the implantable medical device 1105. In one embodiment, the tether retaining features comprise titanium.

In one embodiment, the openings 1128 allow the tether 1112 to be inserted into the tether retaining features 1114 through the openings 1128 rather than sliding the tether through the bores 1126. In this embodiment, the opening 1128 allows insertion of the tether 1112 when a predetermined amount of force is applied to push the tether 1112 through the openings 1128 and into the bores 1126, but prevents the tether 1112 from exiting the tether retaining feature 1114 through the openings 1128. In another embodiment, the tether retaining feature 1114 is closed (i.e., does not include an opening 1128).

When the tether 1112 is located within the tether retaining features 1114, it acts against the top surface 1117 of the connector 1110 to retain the connector 1110 proximal to the implantable medical device 1105. In the embodiment shown in FIG. 11, the tether 1112 forces the bottom surface 1116 of the connector 1110 adjacent to the top surface 1130 of the implantable medical device 1105. In another embodiment, the tether 1112 does not force the bottom surface 1116 of the connector 1110 adjacent to the top surface 1130 of the implantable medical device 1105, but the connector 1110 is retained proximal to and loosely coupled with the implantable medical device 1105. In another embodiment, the tether 1112 retains the connector 1110 proximal to any other surface of the implantable medical device 1105.

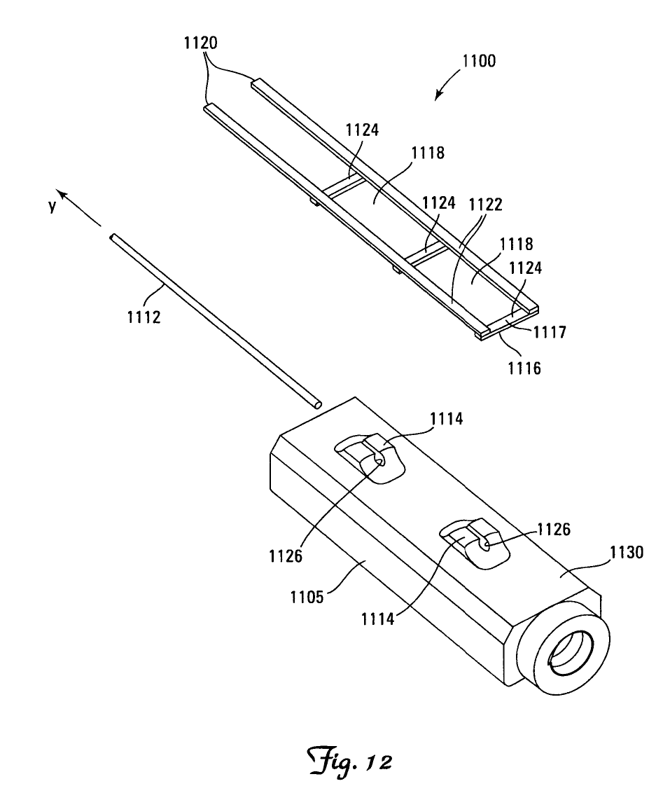

FIG. 12 is an illustration of the delivery system 1100 after the tether 1112 is removed from the tether retaining features 1114. As shown in FIG. 12, once the tether 1112 is slid in a direction Y proximal from the connector 1110, the connector 1110 can be separated from the implantable medical device 1105.

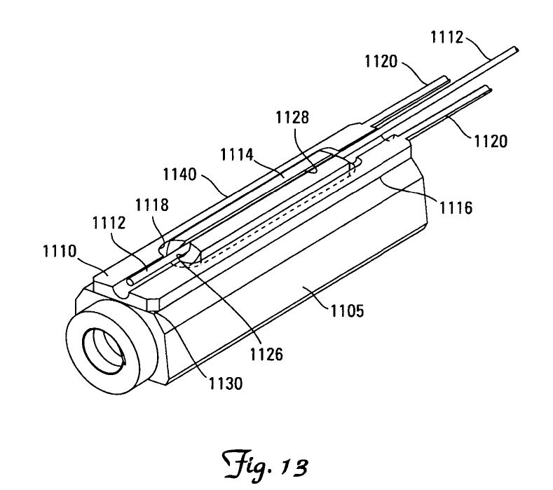

FIG. 13 is a perspective view of another embodiment of the delivery system 1110. In the embodiment shown in FIG. 13, the connector 1110 comprises a plate 1140. The rails 1120 extend in a proximal direction from the plate 1140. The plate 1140 includes an aperture 1118 and the implantable medical device 1105 includes a tether retaining feature 1114. The tether retaining feature 1114 includes a bore 1126 and an opening 1128, but as discussed with respect to FIGS. 11-12, in other embodiments, the tether retaining feature 1114 does not include an opening 1128. The tether 1112, tether retaining feature 1114, and connector 1110 act to keep the bottom surface 1116 of the connector 1110 proximal to the top surface 1130 of the implantable medical device 1105 in the manner discussed with respect to FIGS. 11 and 12. In one embodiment, the plate 1140 is comprised of stainless steel or nitinol. In one embodiment, the plate 1140 has a thickness of about 0.368 inch. In one embodiment, the aperture 1118 has a length of about 0.388 inch and a width of 0.087 inch. In one embodiment, the aperture is about 0.282 inch long and about 0.046 inch wide.

In other embodiments, the delivery system 1100 includes any number of tether retaining features 1114 and apertures 1118. For example, the connector 1110 shown in FIGS. 11 and 12 could have one aperture 1118 and the implantable medical device 1105 could have one tether retaining feature 1114, or the connector 1110 could have a plurality of apertures 1118 and the implantable medical device 1105 could have a plurality of tether retaining features 1114. Similarly, a plate 1140 could include any number of apertures 1118 and the implantable medical device 1105 could have any number of tether retaining features 1114. In other embodiments, the number of apertures 1118 is not the same as the number of tether retaining features 1114. In another embodiment, the connector 1110 has any shape that interlocks with a tether retaining feature 1114 and can be retained proximal to the implantable medical device 1105 using the tether 1112.

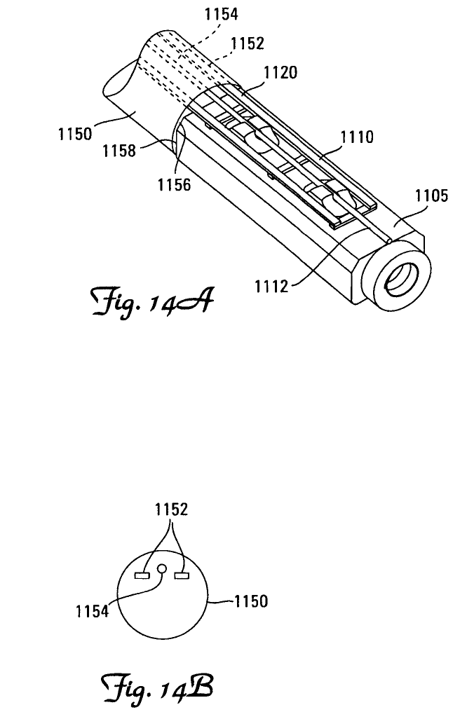

FIGS. 14A-B illustrate an inner member 1150 in conjunction with the implantable medical device 1105, connector 1110, and tether 1112 according to one embodiment of the present invention. The inner member 1150 includes rail lumens 1152, a tether lumen 1154, and a leading face 1156. The rail lumens 1152 are sized to slideably receive the rails 1120 and the tether lumen 1154 is sized to slideably receive the tether 1112. The leading face 1156 acts against the trailing face 1158 of the implantable medical device 1105 to position the implantable medical device 1105 at a desired location within the patient's body. In one embodiment, there is a gap between the leading face 1156 and the trailing face 1158. In one embodiment, the gap is about 0.25 inch. In one embodiment, the rails 1120 are coupled to the inner member 1150 to prevent movement of the rails 1120 with respect to the inner member 1150. In one embodiment, the rails 1120 are coupled to the inner member 1150 at a proximal end (not shown). In one embodiment, the rails 1120 extend to a proximal end (not shown) of the inner member 1150. In another embodiment, the rails 1120 extend a portion of the length of the inner member 1150. In yet another embodiment, the connector 1110 does not include the rails 1120, and instead is coupled to the inner member 1150.

In the end view shown in FIG. 14B, the inner member 1150 is substantially solid and includes the rail lumens 1152 and tether lumen 1154. In other embodiments, the inner member 1150 may include additional lumens, or may comprise a substantially hollow member that receives the rails 1120 and tether 1112. In one embodiment, the inner member 1150 comprises a catheter having a polytetrafluoroethylene (PTFE) or fluoronated ethylene propylene (FEP) inner lining, a 304 V stainless steel braiding, and an outer jacket of Pebax and/or Nylon.

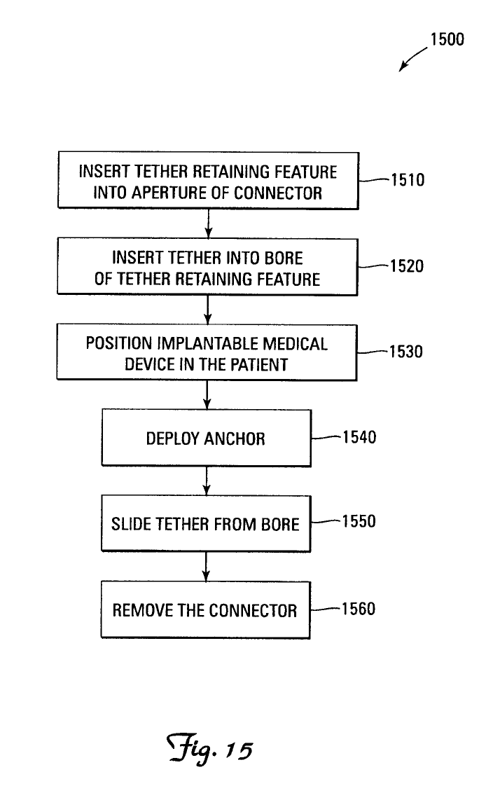

FIG. 15 illustrates an exemplary method 1500 of using the delivery system 1110 according to one embodiment of the present invention. A tether retaining feature 1114 is inserted into an aperture 1118 of a connector 1110 (block 1510). A tether 1112 is inserted into the bore 1126 of the tether retaining feature 1114, thereby retaining the connector 1110 proximal to the implantable medical device 1105 (block 1520). The implantable medical device 1105 is positioned within a patient (block 1530). An anchor coupled to the implantable medical device 1105 is deployed to retain the implantable medical device 1105 at a desired location within the patient (block 1540). In one embodiment, the anchor has the form of the anchor structures disclosed in this application or in previously incorporated U.S. Provisional Patent Application No. 60/844,821, entitled “ANCHOR FOR AN IMPLANTABLE SENSOR.” The tether 1112 is slid from the bore 1126 of the tether retaining feature 1114, thereby releasing the connector 1110 from the implantable medical device 1105 (block 1550). The connector 1110 is removed from the body (block 1560). In one embodiment, the method further comprises delivering the implantable medical device 1105 through an elongated catheter having an inner lumen sized to slideably receive the implantable medical device 1105.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A delivery system for an implantable medical device including a tether retaining feature having a bore, the delivery system comprising:
a connector having a top surface, a bottom surface, a rail extending in a proximal direction from the connector, and an aperture sized to receive the tether retaining feature and reduce movement of the connector with respect to the implantable medical device in a plane parallel to the aperture; and
a tether sized to fit within the bore of the tether retaining feature;
wherein the tether acts against the top surface of the connector to retain the bottom surface of the connector proximal to the implantable medical device when the tether is located within the bore of the tether retaining feature.

2. The system of claim 1 wherein the rail comprises a plurality of rails extending proximally from the connector.

3. The system of claim 2 wherein the connector comprises a cross piece and a plurality of side pieces, and the plurality of side pieces is integral with the plurality of rails.

4. The system of claim 3 wherein the connector is comprised of flat ribbon wire.

5. The system of claim 4 wherein the flat ribbon wire has a width of approximately 0.010 inch and a thickness of approximately 0.005 inch.

6. The system of claim 3 wherein the connector is comprised of a round wire.

7. The system of claim 6 wherein the round wire has a diameter of approximately 0.007 inch.

8. The system of claim 1 wherein the connector comprises a plate.

9. The system of claim 1 wherein the connector includes a plurality of apertures and the implantable medical device includes a plurality of tether retaining features.

10. The system of claim 1 wherein the tether retaining feature includes an opening configured to allow insertion of the tether into the bore through the opening.

11. The system of claim 1 wherein the tether retaining feature is closed and the tether must be slid into the bore.

12. The system of claim 1 wherein the fit of the tether with the tether retaining feature is an interference fit.

13. The system of claim 1 wherein the fit of the tether with the tether retaining feature is loose.

14. A delivery system for an implantable medical device including a tether retaining feature, the delivery system comprising a connector having an aperture sized to receive the tether retaining feature and a tether sized to fit within the tether retaining feature to releasably couple the connector to the implantable medical device.

15. The system of claim 14 further comprising a rail extending in a proximal direction from the connector.

16. The system of claim 14 further comprising an inner member having a leading face located proximal to a trailing face of the connector, wherein the inner member is configured to push the implantable medical device and includes a tether lumen sized to slideably receive the tether and a rail lumen sized to slideably receive the rail.

17. The system of claim 16 wherein the rail includes a proximal end coupled to the inner member.

18. The system of claim 16 wherein the rail comprises a plurality of rails and the inner member includes a plurality of rail lumens.

19. A method for delivering an implantable medical device including a tether retaining feature, the method comprising:
inserting the tether retaining feature into an aperture of a connector;
releaseably coupling the connector to the implantable medical device by inserting a tether into the tether retaining feature;
positioning the implantable medical device within a patient;
deploying an anchor coupled to the implantable medical device;
releasing the connector from the implantable medical device by sliding the tether through a bore of the tether retaining feature; and
removing the connector.

20. The method of claim 19 wherein the method further comprises delivering the implantable medical device through an elongated catheter having an inner lumen sized to slideably receive the implantable medical device.
